(19) United States
(12) Patent Application Publication
(10) Pub. No.: US 2021/0030481 A1
(43) Pub. Date: Feb. 4, 2021

(54) Scanning Apparatus For Scanning An Anatomical Region

(71) Applicant: Navbit IP Pty Ltd, North Sydney (AU)

(72) Inventors: William L. Walter, North Sydney (AU); Daniel Marsden-Jones, North Sydney (AU)

(21) Appl. No.: 16/641,074

(22) PCT Filed: Aug. 20, 2018
(86) PCT No.: PCT/AU2018/050882

§ 371 Date: Feb. 21, 2020

(30) Foreign Application Priority Data
Aug. 22, 2017 (AU) 2017903384

(51) Int. Cl. A61B 34/20; A61B 34/10; A61B 90/00

(52) U.S. Cl. CPC A61B 34/20; A61B 34/10; A61B 90/37; A61B 2090/365; A61B 2034/105; A61B 2090/373; A61B 2034/2048

(57) ABSTRACT

Scanning apparatus is described that includes a scanning device and a processor. The scanning device includes a scanner to scan an anatomical region to obtain scan data and at least one movement sensor to monitor movement of the scanning device to obtain movement data. The processor determines a model of the anatomical region from the scan data and determines placement of the anatomical region from the movement data.

### CROSS-REFERENCE TO RELATED APPLICATION

The present application claims priority to Australian provisional patent application no. 2017903384, filed 22 Aug. 2018, the entire content of which is hereby incorporated by reference.

### TECHNICAL FIELD

The present disclosure relates to surgical navigation and/or computer-assisted surgery in which a model of a region of a patient's anatomy is determined and surgical guidance is provided on the basis of the model.

### BACKGROUND

Surgical navigation commonly requires the determination of an accurate model of a region of a patient's anatomy, such as a bone surface. This can be conducted through a number of medical imaging techniques where an anatomical region to be operated is scanned to obtain scan data and the scan data is processed. On the basis of the processed scan data, surgical guidance can be provided.

Typically, scan data is obtained using medical imaging techniques including CT, MRI, x-rays and ultrasound. While these imaging techniques can provide for an accurate model of a specific anatomical region, they do not necessarily provide a clear indication of the relationship between the anatomical region and anatomical features associated with that anatomical region, such as the placement of the anatomical region relative to an anatomical reference frame and/or the bounds of movement of the anatomical region around one or more joints.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

In one aspect, the present disclosure provides scanning apparatus comprising:

a scanning device, the scanning device comprising:
a scanner to scan an anatomical region to obtain scan data; and
at least one movement sensor to monitor movement of the scanning device to obtain movement data; and

a processor to determine a model of the anatomical region from the scan data and determine placement of the anatomical region from the movement data.

In another aspect, the present disclosure provides a method of scanning an anatomical region comprising:
using a scanner of a scanning device to scan an anatomical region to obtain scan data; and
using at least one movement sensor of the scanning device to monitor movement of the scanning device to obtain movement data; and
determining a model of the anatomical region from the scan data and determining placement of the anatomical region from the movement data.

In a third aspect, the present disclosure provides a method of scanning an anatomical region comprising:

receiving scan data from a scanning device that scans an anatomical region using a scanner to obtain the scan data; and

receiving movement data from at least one movement sensor of the scanning device that monitors monitor movement of the scanning device to obtain movement data; and

determining a model of the anatomical region from the scan data and determining placement of the anatomical region from the movement data.

In yet another aspect, the present disclosure provides software that, when installed on a processor, causes the processor to perform the method of the third aspect.

In yet another aspect, the present disclosure provides a processor and a non-transitory computer-readable memory medium, the non-transitory computer-readable memory medium comprising instructions that cause the processor to carry out the method of the third aspect.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The model may be a virtual model, details of which may be stored by the processor and/or presented on a display. The model may be a two-dimensional or three-dimensional (3D) model, for example.

The anatomical region may be any anatomical region in relation to which a detailed model and/or surgical guidance is desired and can include, for example, a part of a bone such as a bone surface or a part of skin, tissue, muscle, tendon, nail, organ, tooth, ligament and/or cartilage. The anatomical region may also include surgical items such as a drape, a pin, a bone screw, a fiducial marker (e.g. an ECG dot), the tip of a navigation probe, a surgical jig, a saw, a surgical instrument or otherwise. The apparatus and methods of the present disclosure may be used in a variety of different medical and/or surgical procedures and situations including, for example, orthopedic surgery such as knee replacement surgery or other joint-based surgery. The apparatus and methods may enable a surgeon to recognize and understand the alignment of an anatomical region including kinematic alignment and anatomic alignment. The apparatus and methods may enable a surgeon to perform improved surgical procedures such as constitutional varus reconstruction or creation of an angled joint line, for example.

The scanning device may be adapted to be fixed at one or more positions relative to the anatomical region. The apparatus may comprise a mount to fix the scanning device relative to the anatomical region. The mount may fix the scanning device directly to the anatomical region or a surrounding region. For example, when the anatomical region is a part of a bone, the mount may fix the scanning device directly to the bone or tissue surrounding the bone.

The scanning device may comprise a housing. The scanner and the at least one movement sensor may each be at least partially located in the housing. The at least one movement sensor and the scanner may be provided in a fixed positional relationship and may be inseparable during normal use.

The processor may be comprised in the scanning device and may be located, for example, at least partially in the housing. Alternatively, all or part of the processor may be located externally to the scanning device and may communicate with the scanning device via a wired or wireless link. The scanning device may therefore comprise a transmitter and/or receiver for remote communication with all or part of the processor.

It will be recognised that a processor as disclosed herein may comprise a number of control or processing modules for controlling one or more functions of the apparatus and methods and may also include one or more storage elements, for storing data, e.g., scan data, movement data and/or model data. The modules and storage elements can be implemented using one or more processing devices and one or more data storage units, which modules and/or storage devices may be at one location or distributed across multiple locations and interconnected by one or more communication links. Processing devices that are used may be located in desktop computers, laptop computers, tablets, smartphones, personal digital assistants and other types of processing devices, including devices manufactured specifically for the purpose of carrying out functions according to the present disclosure.

Further, the processing modules can be implemented by a computer program or program code comprising program instructions. The computer program instructions can include source code, object code, machine code or any other stored data that is operable to cause the processor to perform the steps described. The computer program can be written in any form of programming language, including compiled or interpreted languages and can be deployed in any form, including as a stand-alone program or as a module, component, subroutine or other unit suitable for use in a computing environment. The data storage device(s) may include suitable computer readable media such as volatile (e.g., RAM) and/or non-volatile (e.g., ROM, disk) memory or otherwise.

The determined placement of the anatomical region may comprise one or more of a position and orientation of the anatomical region. The determined placement of the anatomical region may be the placement relative to a reference frame. The monitoring of movement of the scanning device may enable a placement of the scanning device to be determined relative to the reference frame. The placement of the anatomical region within the reference frame may be determined from the placement of the scanning device within the reference frame. The placement of the anatomical region within the reference frame may be determined from a combination of the determined placement of the scanning device within the reference frame and further from (i) the scan data, such as recorded time of flight for a scanning signal transmitted to and from the anatomical region and/or (ii) an externally observed physical relationship between the scanning device and the anatomical region, e.g. if the scanning device is placed at a known or predetermined distance from the anatomical region.

In one embodiment, to enable placement of the scanning device to be determined relative to the reference frame, the scanning device is moved between a registration position and a scanning position. At the scanning position, the scanning device may scan the anatomical region. At the registration position, the placement of the scanning device may be registered to the reference frame. The registration of the scanning device may produce registration data indicative of the placement of the scanning device within the reference frame. The placement of the registration device may be ‘zeroed’ at the registration position or otherwise recorded.

The scanning device may be positioned at the scanning position, to perform the scan of the anatomical region, before or after the registration of the scanning device at the registration position. During movement of the scanning device from the registration position to the scanning position, or during movement of the scanning device from the scanning position to the registration position, the change in placement of the scanning device may be monitored and, from the monitoring, the movement data is acquired. From the movement data, and the registration data, the placement of the scanning device at the scanning position, within the reference frame, can be determined.

The scanning device may be positioned at the registration position by being fixed to a mount that has a known relationship with the reference frame. For example, surfaces of the mount may be aligned with anatomical axes on which the reference frame is based and fixing of the scanning device to the mount may align the scanning device to the anatomical axes. In some embodiments, the mount may be fixed to a pelvis of the body.

In some embodiments, the scanning device may be positioned at the scanning position by being fixed to a mount. Alternatively, the scanning device may be positioned at the scanning position in a hand-held manner. A surgeon may hold the scanning device during scanning of the anatomical region, for example.

In some embodiments, the monitoring of movement by the scanning device may be used to determine the reference frame, in addition to determining the placement of the scanning device within the reference frame.

For example, in one embodiment, the scanning device is fixed relative to the anatomical region that is scanned. While the scanning device is fixed relative to the anatomical region, the anatomical region is moved and the at least one movement sensor of the scanning device monitors the movement of the anatomical region. The reference frame may be determined from the movement. In one example, the reference frame may be aligned to the centre of rotation of the anatomical region. For example, the origin of the reference frame may be placed at the centre of rotation of the anatomical region. During movement of the anatomical region, the anatomical region may rotate about the centre of rotation, enabling the at least one movement sensor to obtain movement data that can be used to determine the location of the centre of rotation of the anatomical region and thus the frame of reference. The movement data may indicate the arc of movement of the scanning device, from which arc the centre of rotation can be determined and also a distance of the scanning device from the centre of rotation. Thus, both a frame of reference based on the centre of rotation can be determined, in addition to the placement of the scanning device within that frame of reference, by virtue of the monitored movement of the anatomical region.

When the reference frame is based on the centre of rotation of the anatomical region, the reference frame may be further based on an axis extending between the centre of rotation and an anatomical landmark on the anatomical region that is identified from the scan data. For example, if the anatomical region that is scanned is the distal surface of the femur, the landmark may be the midpoint between the two femoral condyles. Rotation around this axis may be fixed by the condylar axis or the sulcus line, for example, enabling three orthogonal axes to be determined.

In some embodiments, the movement data may be obtained while the scanning device is in the scanning position. Accordingly, the scanning device may be fixed relative to the anatomical region in one position only to both determine placement within a reference frame and to scan the anatomical region. In such instances, no movement to or from any separate registration position may be required.

When the scanning device is located at a registration position, in some embodiments, monitoring of movement by the scanning device may be used to determine the frame of reference to which the scanning device is registered at the registration position. For example, when the scanning device is fixed at the registration position, e.g. when it is mounted to a bone such as the pelvis, the body may be rotated between different anatomical configurations (e.g. between a supine position and a prone position, etc.) and the movement data acquired from this rotation may be used to determine axes or vectors of the body and thus a frame of reference for the body.

In some embodiments, the processor may be configured to place the model of the anatomical region in a virtual scene having a virtual reference frame. The model of the anatomical region may be placed in the virtual scene relative to a virtual reference frame in accordance with the determined placement of the actual anatomical region relative to its frame of reference. In some embodiments, the virtual scene may comprise a virtual centre of rotation of the anatomical region, the model of the anatomical region being placed relative to the virtual centre of rotation of the anatomical region based on the determined placement of the actual anatomical region relative to the centre of rotation of the anatomical region. The virtual scene may comprise dynamic information indicative of the way the anatomical region moves. For example, the virtual scene may allow the model of the anatomical region to be rotated about the virtual centre of rotation, for example, or otherwise manipulated. The virtual scene may be presented on a display.

The scanning apparatus may be used in combination with a pre-operative model. The pre-operative model may include a scan such as a CT scan, an MM or otherwise, of the anatomical region. Through scanning of the anatomical region and the determining of the placement of the anatomical region, the scanning apparatus can be used to register the position of a corresponding pre-operative model of the anatomical region in a virtual scene. Thus, in addition or as an alternative to controlling, based on the scan and movement data, the positioning within a virtual scene of a model of the anatomical region that has been determined using the scanning apparatus, the processor may control, based on the scan and movement data, the positioning within a virtual scene of a pre-operative model of the anatomical region.

As indicated, the virtual scene including the model may be presented on a display. The display may be a computer monitor, a TV screen, a projector or otherwise. In some embodiments, the display may be mounted to a user. For example, the display may be comprised in wearable computer glasses also known as smartglasses. Using the wearable computer glasses or otherwise, the virtual scene may be presented to a user as part of mixed reality view. The mixed reality view may provide for surgical navigation guidance. In this regard, it may include a pre-operative plan for surgery.

When the apparatus is used to determine a centre of rotation for an anatomical region, the scanning position may be distal to the centre of rotation. For example, if the anatomical region is a bone surface, the bone surface may be a distal bone surface, at an opposite end of the bone from the centre of rotation of the bone. Thus, the scanner of the scanning device may be adapted to scan a distal end surface of a bone. At the scanning position, the scanning device may be fixed adjacent the distal end surface of the bone.

As indicated, the anatomical region may be a part of a bone such as a bone surface. The bone may be a femur and the surface scanned by the scanner may comprises one or more of a femoral condyle surface and a patellofemoral groove surface. Alternatively, the bone may be a tibia and the surface scanned by the scanner may comprise a surface of the tibial plateau. Nevertheless, the apparatus may be used to scan a variety of different bone surfaces of a variety of different bones, including long bones, such as the humerus, radius, ulna, metacarpus, phalanges of the hand, femur, tibia, fibula, metatarsus and phalanges of the feet, or otherwise. The anatomical region is not necessarily a part of a bone, however. For example, as indicated above it may be a surface of a tooth, a skin surface, a surface of an organ, a nail surface or otherwise.

When the anatomical region is a surface of the femur, the processor may be configured to determine, for example, from the determined model of the anatomical region and the placement, one or more of:

femoral anatomic coronal plane alignment;

distal femoral rotation; and

femoral size.

When the anatomical region is a surface of the tibia, the processor may be configured to determine, for example, from the determined model of the anatomical region and the placement, one or more of:

tibial coronal plane alignment;

tibial sagittal plane alignment;

tibial proximal plane;

tibial rotation; and

tibial component size.

The scanner may be any type of scanner suitable to obtain scan data that can be used to prepare a model of an anatomical region, such as a 3D scanner to obtain a 3D model of an anatomical region. For example, the scanner may be a laser scanner. As another example, the scanner may be an ultrasound scanner.

When the scanner is a laser scanner, the model of the anatomical region may be determined based on a triangulation scanning method, a phase shift scanning method, or a time of flight scanning method. In some embodiments, the laser scanner may emit structured light.

The at least one movement sensor may comprise any one or more of the same or different types of movement sensors. For example, the at least one movement sensor may comprise a gyroscope. Additionally or alternatively, the at least one movement sensor may comprise an accelerometer. Additionally or alternatively, the at least one movement sensor may comprise a magnetometer. The movement data may be derived from any one or combination of movement sensors such that movement of the scanning device in two or three-dimensional space can be identified.

The bone scanning apparatus may comprise a guide to automatically guide a surgical procedure relative to the anatomical region, based on the model of the anatomical region and the determined placement of the anatomical region. The guide may automatically guide positioning of a drill hole or a cut in the anatomical region, for example. The guide may interact directly with the anatomical region. The guide may comprise a laser that shines light on the anatomical region to guide the surgical procedure, for example. Additionally or alternatively, the guide may comprise a drill guide or cutting jig that is movable relative to the anatomical region to guide the surgical procedure. The scanning apparatus may comprise a display, wherein the guide comprises information presented on the display. The information presented on the display may include an image of the anatomical region and markers or other features acting as guides. As indicated above, the display may be comprised in wearable glasses or otherwise.

### BRIEF DESCRIPTION OF DRAWINGS

By way of example only, embodiments of the present disclosure are now described with reference to the accompanying drawings in which:

FIG. 1 shows a side view of scanning apparatus according to an embodiment of the present disclosure;

FIG. 2 shows a schematic view of components of a scanning device of the apparatus of FIG. 1;

FIG. 3 shows a side view of the scanning apparatus of FIG. 1 mounted to a femur and operated to determine a model and placement of an anatomical region in a manner according to one embodiment of the present disclosure;

FIG. 4 represents a displayed model of an anatomical region that is obtained using the scanning apparatus of FIG. 1;

FIG. 4a shows a user wearing computer glasses that display a model to the user in accordance with an embodiment of the present disclosure, e.g., for the purpose of mixed reality surgical navigation;

FIGS. 5a to 5d show side views of the scanning apparatus of FIG. 1 mounted to a femur and operated to determine a model and placement of an anatomical region in a manner according to further embodiments of the present disclosure; and

FIG. 6 shows a schematic view of components of a scanning device of scanning apparatus according to another embodiment of the present disclosure.

### DESCRIPTION OF EMBODIMENTS

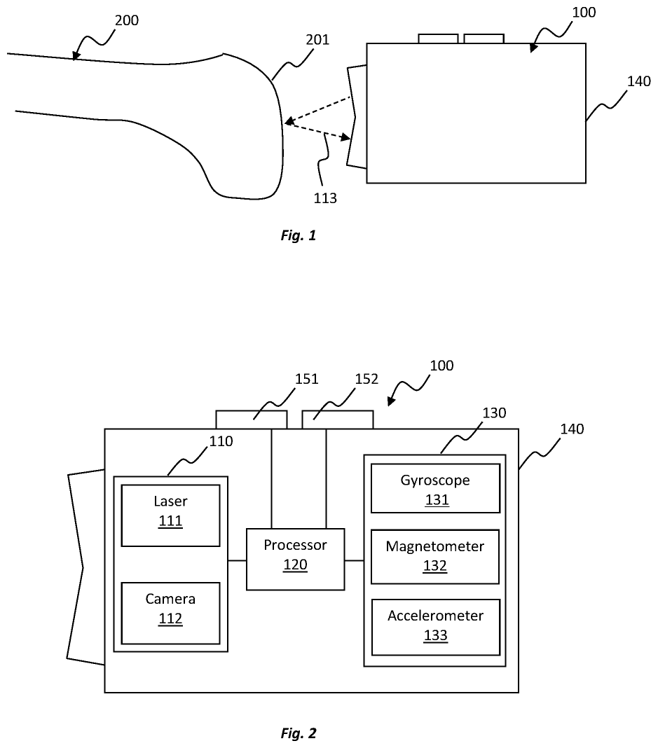

Scanning apparatus according to an embodiment of the present disclosure is illustrated in FIGS. 1 and 2. The scanning apparatus includes a scanning device 100, which scanning device 100 includes a scanner 110, a processor 120 and at least one movement sensor 130. In this embodiment, three different movement sensors are provided, a gyroscope 131, a magnetometer 132 and an accelerometer 133.

The scanner 110 is provided to scan an anatomical region. A model of the anatomical region can be determined from the scan data. The model may be recorded by the processor and optionally presented on a display.

In relation to the embodiments now described, bone surfaces are provided as examples of anatomical regions that are scanned and modelled. However, the present apparatus may be adapted for use with a variety of different anatomical regions, which regions may comprise bone/bone surface and/or other features such as one or more of skin, tissue, muscle, tendon, nail, organ, tooth, ligament, cartilage or a surgical item, such as a drape, a pin, a bone screw, a tracker, a fiducial marker (e.g. an ECG dot), the tip of a navigation probe, a surgical jig, or a surgical instrument. Where, for example, a surgeon is seeking to prepare of a femur from a posterior approach, the anatomical region may comprise the bone of the proximal femur, the lower leg and foot wrapped in drapes. Where, for example, a tracker or fiducial marker is comprised in the anatomical region, it may be positioned to mark a particular anatomical landmark. As an example, an ECG dot may be placed on a medial malleolus. Where, for example, the tip of a probe is comprised in the anatomical region, it may be touching an anatomical landmark. Where surgical instruments are comprised in the anatomical region, methods according to the present disclosure may be used to determine if the instruments are at an appropriate location/orientation relative to other features comprised in the anatomical region, such as a bone surface.

In the embodiment illustrated in FIGS. 1 and 2, the scanner includes a laser 111 and a camera 112. The laser 111 is adapted to shine laser light on the anatomical region, and specifically the bone surface, which laser light is reflected off the bone surface and received by the camera 112, generally as represented by arrows 113 in FIG. 1. The laser light is movable to scan across the bone surface. In general, a number of different scanning techniques, that are known by the person skilled in the art, may be used in embodiments of the present disclosure, whether using laser light, cameras or otherwise, in order to obtain scan data that is indicative of the shape and/or configuration of the bone surface (or other anatomical region). The scanner may rely on a triangulation scanning method, a phase shift scanning method, or a time of flight scanning method, for example. In some embodiments, the laser scanner may emit structured light. The scanner may be a laser scanner or an ultrasound scanner, for example. The scan data may be produced directly from the scanner 110 or produced at least in part by the processor 120 that is connected to the scanner 110.

The scanning device 100 includes a housing 140. In this embodiment, the scanner 110, the processor 120 and the movement sensor 130 are all located in the housing 140. Moreover, at least the scanner 110 and the movement sensor 130 have a fixed positional relationship to each other. A button 151 is provided in the scanning device 100, the button 151 being accessible to the outside of the housing 140. The button 151 is operationally connected to the scanner 110, e.g., via the processor 120, and is operable by a user to trigger the scanning of the bone surface by the scanner 110. As an alternative to a button 151, other types of actuation devices such as a slider, a touch sensitive pad, or a remote control may be used.

In FIG. 2, the processor 120 is illustrated as a discrete element of the scanning device 100 that is connected to the scanner 110 and the movement sensor 130 via communication links. Nevertheless, in some embodiments, the processor may be integrated with the scanner 110 and/or the movement sensor 130. Additionally or alternatively, all or part of the processor may be located externally to the scanning device 100 and may communicate with the scanning device 100 via one or more wired or wireless links. The scanning device 100 may comprise a transmitter and/or receiver for remote communication with all or part of the processor.

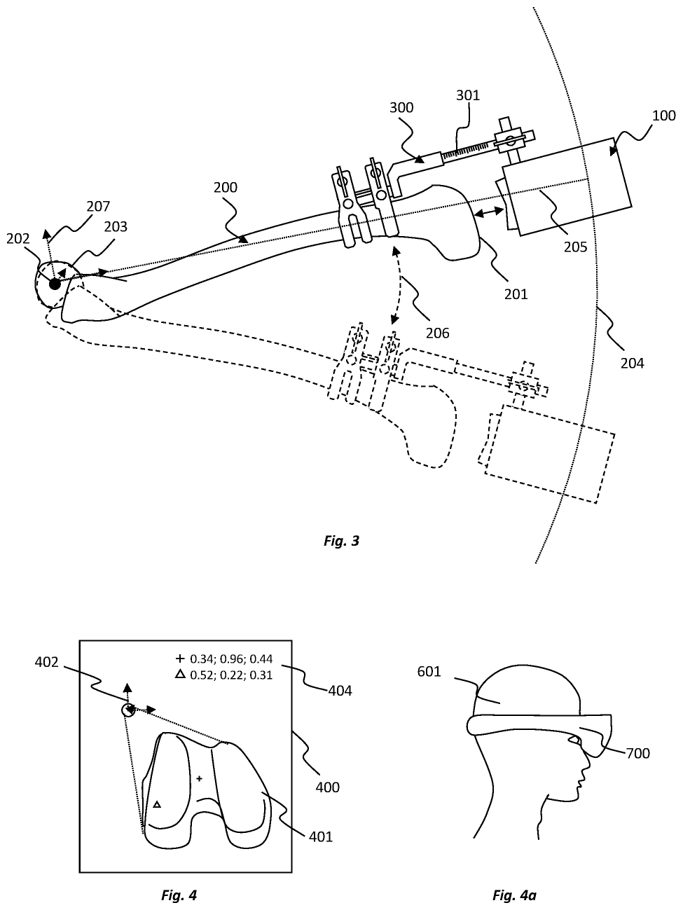

The scanning device 100 as illustrated in FIG. 1 is positioned in a scanning position for scanning the bone surface, and particularly, in this example, at a scanning position where it is adjacent to and oriented to scan a distal bone surface 201 of a femur 200. The scanning device 100 can be fixed relative to the bone surface at the scanning position by being mounted to bone or to tissue surrounding bone, for example. The apparatus of the present disclosure may therefore include a mount in addition to the scanning device 100. An example of a mount 300, that fixes the scanning device 100 relative to a bone surface 201 is illustrated in FIG. 3. The mount 300 is an adjustable mount, enabling the position of the scanning device 100 to be adjusted and fixed in a controlled manner relative to the bone surface 201, although a non-adjustable mount may also be used. Moreover, in some embodiments, the scanning device may instead be hand-held at the scanning position or supported at the scanning position by additional apparatus that does not connect or mount to bone or the body.

The movement sensor 130 of the scanning device 100 is used to monitor movement of the scanning device 100 to obtain movement data. The movement data may be derived from any one or combination of the gyroscope 131, magnetometer 132 and accelerometer 133, such that movement of the scanning device 100 in three-dimensional space can be identified. The movement data can be used to determine placement of the bone surface. The determined placement of the bone surface can be associated with the model of the bone surface that is obtained using the scan data. The placement of the bone surface can be determined relative to a reference frame. In some embodiments, the reference frame may also be determined from the movement data.

In one embodiment, as now described with reference to FIG. 3, while the scanning device 100 is fixed relative to the bone 200 in a scanning position, movement data is obtained to determine a reference frame, to determine the placement of the scanning device relative to the reference frame, and, from this, to determine the placement of the scanned bone surface relative to the reference frame.

To determine the reference frame in this embodiment, while the scanning device 100 is fixed relative to the bone 200 in the scanning position, the bone 200 (and therefore the scanning device 100) is rotated about the centre of rotation 202 of the bone 200, as indicated by arrow 206 and broken-line illustrations in FIG. 3. Since the bone 200 is a femur in this example, the centre of rotation 202 is located at the head 203 of the femur/the hip joint. While being rotated, the movement sensor 130 of the scanning device 100 monitors the rotational movement and provides movement data to the processor 120. The rotation is generally in an arc 204 around the centre of rotation 202 of the bone 200. The orientation and the radius 205 of the arc 204 are therefore indicative of the location of, and distance to, the centre of rotation 202, respectively. This information, contained within the movement data, can be used by the processor 120 to determine the reference frame. An example reference frame is illustrated in FIG. 3 by the axes 207. The origin of the reference frame 207 is at the centre of rotation 202 and the reference frame 207 is further defined by an axis extending between the centre of rotation 202 and an anatomical landmark on the bone, which landmark may be identified from scan data. For example, when the bone surface that is scanned is the distal surface 201 of the femur 200 as illustrated in FIG. 3, the landmark may be a midpoint that is identified between the two femoral condyles. A virtual rotation around this axis may be fixed by the condylar axis or the sulcus line, for example, enabling three orthogonal axes of the reference frame 207 to be determined.

In this embodiment, the approach to determining the reference frame 207 using the scanning device 100 inherently determines the placement of the scanning device 100 relative to the reference frame 207. The placement of the scanned bone surface 201 relative to the reference frame 207 can be determined from a combination of the determined placement of the scanning device 100 relative to the reference frame 207 and the placement of the scanning device 100 relative to the bone surface 201. As illustrated in FIG. 3, a placement of the scanning device 100 relative to the bone surface 201 can be externally observed, e.g. by using calibration marks 301 on the mounting device 300 and/or by using other instruments such as callipers or rulers. Alternatively, the placement can be determined from the scan data. For example, the scan data may include time of flight information for a scanning signal transmitted to and from different regions of the bone surface, which is indicative of the placement of the bone surface relative to the scanning device.

With reference to FIG. 4, in this embodiment, the processor 120 is configured to place the model 401 of the bone surface, that is determined from the scan data, in a virtual scene 400 having a virtual reference frame 402, the placement of the model 401 within the virtual scene/virtual reference frame 400, 402 being in accordance with the determined placement of the actual bone surface 201 relative to the frame of reference 207 based on the movement data, as discussed above. The virtual scene 400 can be presented on a display, for example. In this embodiment, since the virtual frame 402 is based on a centre of rotation of the bone and its bone surface 201, the virtual scene may allow the model 402 of the bone surface to be rotated about the virtual centre of rotation upon interaction by a user. Moreover, the virtual scene 400 may provide for information to the user relating to the model, e.g. coordinate data 404 for points on the model within the reference frame. The virtual scene is not necessarily displayed to the user, however. The virtual scene may be recorded or stored by the processor and used to provide guidance to a user by other means.

In any embodiments, the display may be a computer monitor, a TV screen, a projector or otherwise. In some embodiments, the display may be mounted to a user. For example, as represented in FIG. 4a, the display may be comprised in wearable computer glasses 700 also known as smartglasses. Using the wearable computer glasses 700 or otherwise, the virtual scene may be presented to a user 601 as part of mixed reality view. The mixed reality view may provide for surgical navigation guidance. In this regard, it may include a pre-operative plan for surgery.

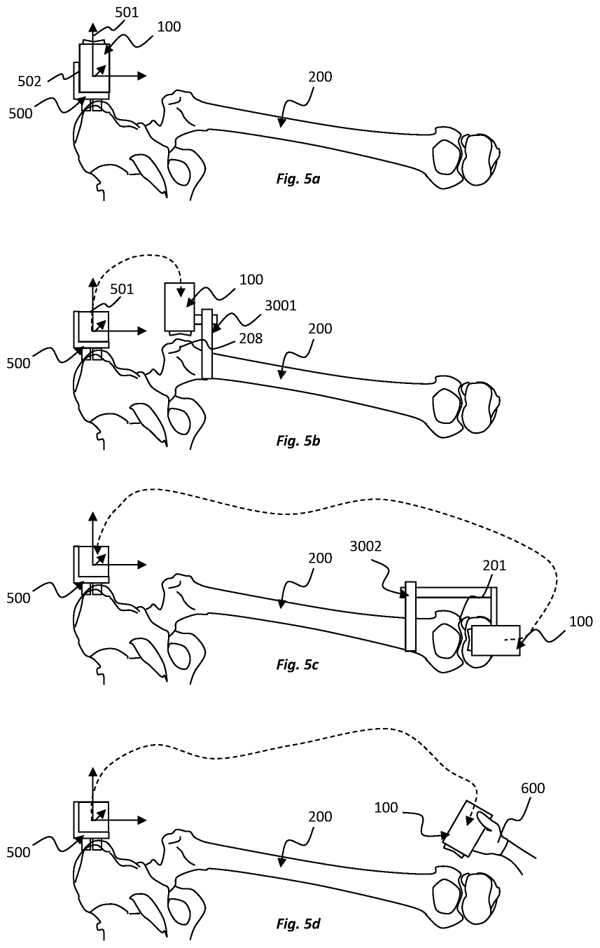

In an alternative embodiment, as now described with reference to FIGS. 5a to 5d, to enable placement of the scanning device 100 to be determined within a reference frame, the scanning device 100 is moved between a registration position and a scanning position.

Again, at the scanning position, the scanning device 100 is configured to scan the bone surface. However, the scanning device is placed in a different, registration position, to register placement of the device 100 relative to the reference frame.

Positioning of the scanning device 100 at a registration position is illustrated in FIG. 5a. In this embodiment, the scanning device 100 is positioned at the registration position by being fixed to a mount 500 that has a known geometrical relationship with the reference frame 501 and specifically, in this embodiment, with a reference frame 501 that is centred generally on or adjacent the pelvis of the patient and oriented in accordance with the anatomical axes of the patient at the pelvis. Surfaces 501 of the mount 500 are aligned with the anatomical axes and fixing of the scanning device 100 to the mount 500 aligns the scanning device 100 to the anatomical axes.

When positioned at the registration position, the scanning device 100 can be registered to the reference frame. Registration can occur, for example, by pressing a button 152 that is provided in the scanning device 100, the button 152 being accessible to the outside of the housing 140. The button 152 is connected to processor 120. As an alternative to a button 152, other types of actuation devices such as a slider, a touch sensitive pad, or a remote control may be used. Additionally or alternatively, an automated actuation device may be triggered through mere fixing of the scanning device 100 to the mount 500, for example. When the button or other actuation device is actuated, it indicates to the processor 120 that the scanning device 100 is in the registration position and registration to the reference frame 501 should take place. Spatial coordinates of the scanning device can be ‘zeroed’ at this time, for example. Based on the registration to the reference frame 501, any movement of the scanning device can be monitored relative to the reference frame 501.

The scanning device 100 can be positioned at the scanning position, to perform the scan of the bone surface, before or after the registration of the scanning device 100 at the registration position. During movement of the scanning device 100 from the registration position to the scanning position, or during movement of the scanning device 100 from the scanning position to the registration position, the change in placement of the scanning device 200 is monitored by the movement sensor 130 and, from that monitoring, the movement data is obtained.

As illustrated in FIG. 5b, for example, after registration of the scanning device 100 at the registration position shown in FIG. 5a, the scanning device 100 can be moved to a scanning position where it is mounted adjacent a bone surface such as the proximal end surface 208 of a femur 200. During this movement, the placement of the scanning device 100 relative to the reference frame 501 is continually monitored so that its placement relative to the reference frame 501 when it arrives at the scanning position, and when the scanning of the bone surface 208 is conducted (e.g. when button 151 is pressed), is known. The placement of the scanned bone surface 208 relative to the reference 501 can be determined from a combination of the determined placement of the scanning device 100 relative to the reference frame 501 and the placement of the scanning device 100 relative to the bone surface 208. The placement of the scanning device 100 relative to the bone surface 208 can be externally observed or determined from scan data, e.g., in a similar manner to that described above.

As indicated, the registration to the reference frame 501 does not need to take place before scanning the bone surface, however. As illustrated in FIG. 5c, for example, after being placed in a scanning position and after scanning a bone surface such as the distal surface 201 of the femur (e.g. when button 151 is pressed), the scanning device 100 can be moved to the registration position illustrated in FIG. 5a. During this movement, the placement of the scanning device 100 is continually monitored, allowing determination of its placement at the scanning position relative to the reference 501 to be extrapolated from the movement data and the subsequent registration of the scanning device 100 to the reference frame 501.

In FIGS. 5b and 5c, the scanning device 100 is fixed at the respective scanning positions using mounts 3001, 3002. However, the scanning device may instead be positioned at the scanning position in a hand-held manner as illustrated in FIG. 5d; a surgeon's hand 600 may hold the scanning device during scanning of the bone surface, for example.

Again, in the embodiments illustrated in FIGS. 5a to 5d, the processor may be configured to place the model of the bone surface in a virtual scene having a virtual reference frame, e.g., in a similar manner to that described with reference to FIG. 4.

In the embodiments discussed above, surfaces of the femur are provided as example bone surfaces that are scanned, including a distal demur surface that can comprise one or more of a femoral condyle surface and a patellofemoral groove surface. Other bone surfaces may be scanned, however, such as the tibial plateau or even surfaces of a variety of other bones such as the humerus, radius, ulna, metacarpus, phalanges of the hand, fibula, metatarsus and phalanges of the feet, or otherwise.

In addition or as an alternative to providing a virtual scene, the processor in some embodiments can be configured to determine anatomical information relating to the bone that is scanned, based on the model of the bone surface and the placement of the bone surface. For example, when the bone that is scanned is the femur, the processor may determine, based on the model of the bone surface and the placement, one or more of: femoral anatomic coronal plane alignment; distal femoral rotation; and femoral size. As another example, when the bone that is scanner is the tibia, the processor may be configured to determine, from the determined model of the bone surface and the placement, one or more of: tibial coronal plane alignment; tibial sagittal plane alignment; tibial proximal plane; tibial rotation; and tibial component size.

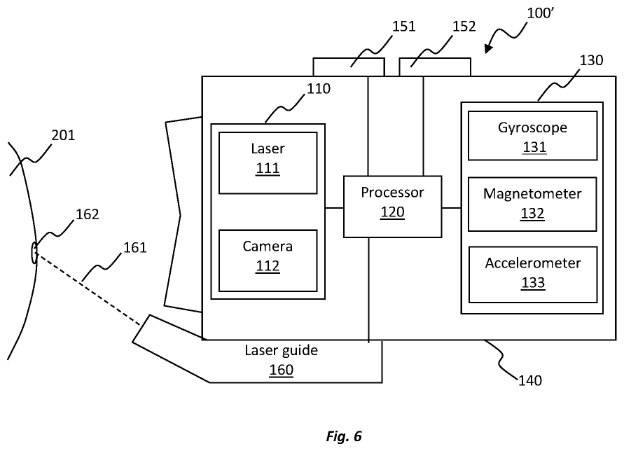

In addition or as an alternative to providing a virtual scene and/or anatomical information, the processor may be configured to determine guidance information based on the model of the bone surface and the placement of the bone surface. The bone scanning apparatus may comprise a guide to automatically guide a surgical procedure relative to the bone surface based on the guidance information. In accordance with this, in one embodiment, as illustrated in FIG. 6, a scanning device 100′ is provided that it similar or identical to the scanning device 100 described above, but which includes, additionally, a guide that automatically guides positioning of a drill hole or a cut in the bone surface based on the determined guidance information. In this embodiment, the guide is a laser guide 160 that interact directly with the bone surface 201, specifically by shining laser light 161 on the bone surface 201 such that there is a light spot 162 on the bone surface indicative of where a drill hole should be made in that surface. A surgeon may permanently mark the bone surface at the location of the light spot 162 for subsequent drilling. Additionally or alternatively, the guide may comprise a drill guide or cutting jig that is movable relative to the bone surface to guide the surgical procedure. The bone scanning apparatus may comprise a display, wherein the guide comprises information presented on the display. The information presented on the display may include an image of the bone surface and markers or other features acting as guides.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. Scanning apparatus comprising:
a scanning device, the scanning device comprising:
a scanner to scan an anatomical region to obtain scan data; and
at least one movement sensor to monitor movement of the scanning device to obtain movement data; and
a processor to determine a model of the anatomical region from the scan data and determine placement of the anatomical region from the movement data.

2. (canceled)

3. The scanning apparatus of claim 1, wherein the anatomical region comprises one or more of bone, skin, tissue, muscle, tendon, nail, organ, tooth, ligament, cartilage or surgical items.

4. The scanning apparatus of claim 1, comprising a mount to fix the scanning device relative to the anatomical region.

5. (canceled)

6. The scanning apparatus of claim 1, wherein the scanning device comprises a housing and wherein the scanner and the at least one movement sensor are each at least partially located in the housing.

7. (canceled)

8. (canceled)

9. The scanning apparatus of claim 1, wherein the determined placement of the anatomical region comprises one or more of a position and orientation of the anatomical region.

10. The scanning apparatus of claim 1, wherein the determined placement of the anatomical region comprises placement of the anatomical region relative to a reference frame.

11. The scanning apparatus of claim 10, wherein the processor is configured to determine the reference frame from the movement data.

12. The scanning apparatus of claim 11, wherein the reference frame is based at least partly on a centre of rotation of the anatomical region.

13. The scanning apparatus of claim 12, wherein the scanning device is configured to be fixed relative to the anatomical region to monitor a movement of the anatomical region about the centre of rotation to obtain the movement data and wherein the processor is configured to determine, from the movement data, the location of the centre of rotation, the reference frame, and the placement of the scanning device relative to the reference frame.

14. The scanning apparatus of claim 13, wherein the processor is configured to determine the placement of the anatomical region relative to the reference frame from the determined placement of the scanning device relative to the reference frame and a known or measured placement of the scanning device relative to the anatomical region.

15. The scanning apparatus of claim 12, wherein the anatomical region comprises a surface of a bone, the centre of rotation being a centre of rotation of the bone.

16. (canceled)

17. The scanning apparatus of claim 16, wherein the surface of the bone is a distal surface of the femur and the centre of rotation is at a hip joint.

18. The scanning apparatus of claim 1, wherein the scanning device is adapted to be placed in a scanning position in which the scanner scans the anatomical region, wherein the movement data is obtained while the scanning device is in the scanning position.

19. The scanning apparatus of claim 1, wherein the scanning device is fixed relative to the anatomical region in one position only to obtain the movement data and the scan data

20. The scanning apparatus of claim 10, wherein the scanning device is adapted to be placed in a registration position to register placement of the scanning device to the reference frame.

21. The scanning apparatus of claim 20, wherein the scanning device comprises a button or other actuation device that is connected to the processor and operated when the scanning device is at the registration position to cause the registration of the scanning device to the reference frame.

22. The scanning apparatus of claim 20, wherein the scanning device is adapted to be placed in a scanning position, separated from the registration position, in which the scanner scans the anatomical region.

23. The scanning apparatus of claim 22, wherein the at least one movement sensor is adapted to monitor movement of the scanning device from the registration position to the scanning position or from the scanning position to the registration position to obtain the movement data and the processor is configured to, based on the movement data, determine the placement of the scanning device relative to the reference frame.

24. The scanning apparatus of claim 23, wherein the processor is configured to determine the placement of the anatomical region relative to the reference frame from the determined placement of the scanning device relative to the reference frame and a known or measured placement of the scanning device relative to the anatomical region.

25. The scanning apparatus of claim 1, wherein the processor is configured to place the model of the anatomical region in a virtual scene the model being oriented in the scene based on the determined placement.

26-49. (canceled)
